(19) United States
(12) Patent Application Publication
(10) Pub. No.: US 2009/0062907 A1
(43) Pub. Date: Mar. 5, 2009

(54) Self-expanding valve for the venous system

(76) Inventors: Rodolfo C. Quijano, Laguna Hills, CA (US); Hosheng Tu, Newport Beach, CA (US)

Correspondence Address: HOSHENG TU, 15 RIEZ, NEWPORT BEACH, CA, 92657-0116, US

(21) Appl. No.: 11/897,794

(22) Filed: Aug. 31, 2007

(51) Int. Cl. A61F 2/06

(52) U.S. Cl. 623/1.24

(57) ABSTRACT

A venous valve device and method of formation are described to provide antegrade blood flow in the deep venous vessels of the leg or in other venous vessels of the body having incompetent or irreversibly dysfunctional valves. The venous valve device is made of a sheet of biocompatible material, comprising a longitudinal wire-frame structure that is a continuous seamless wire loop and plural anchoring mesh-lattice wing members spaced apart and connected to the base of the wire-frame structure.

### FIELD OF THE INVENTION

The present invention generally relates to a self-expanding valve to control the flow of blood in any section of the venous system. More particularly, the present invention relates to a medical device having shape-memory alloys frame with cross-linked decellularized pericardial tissue or polymer or Nitinol membrane as a replacement venous valve.

### BACKGROUND OF THE INVENTION

Various disease conditions of the venous system, from the foot to the groin, can be attributed to partial or total dysfunction of unidirectional valves that are normally found in specific regions of the deep, perforating or superficial veins of the leg. The human leg has both a superficial vein system and a deep vein system that are in fluid communication to each other through a series of perforating veins, each with a perforator vein valve. The superficial short and long saphenous veins carry blood at low pressure and have valves to prevent reversal blood flow, thus maintaining flow of blood in the direction towards the heart. These veins lie outside the deep fascia and drain into the deep venous system comprised of the popliteal and femoral veins. Both of these systems are perforating veins that pass through the fascia.

Blood is returned to the heart from the periphery, via the venous system, by a combination of mechanisms including the compression of the veins by contraction of the leg muscles and diaphragmatic pressure during respiration and intra-thoracic and intra-abdominal pressures. Calf muscles thus, when active, act as a pump, forcing venous blood upwards towards the heart. Healthy valves in the perforating veins prevent reverse flow towards the superficial veins. During periods when muscles are relaxed, blood flows from the superficial to the deep veins below the closed valves, before the calf muscle pump acts again to force the blood away from the limbs. If the perforating valves become incompetent, the reverse pressure is transmitted directly to the superficial venous system, reversing the flow, damaging more distal valves, and eventually leading to varicose veins. Damaged valves in the deep and perforating veins are one cause of chronic venous insufficiency (CVI).

Pressure at different periods of activity or inactivity reflects itself in increases or decreases of pressure immediately above the ankle, in the ‘gait’ area. For instance, when a person is in the decubitus position and relaxed, pressure in that area is in the order of 15 mmHg and the venous valve is mostly open with leaflets apart floating in the blood in parallel direction of flow. While the person is on the move or walking, the muscle pump moves blood upward against gravity and the pressure around the ankle is in the order of 45 mmHg. When the person is seated, the pressure is higher, about 56 mmHg. When standing immobile for periods of time, the pressure right above the ankle is about 85 mmHg. The continuous pressure on this area can cause stagnation of blood, and venous stasis ulcers can develop.

CVI disease in the early stages will evidence development of very tenuous superficial capillary-like veins that can be seen transparently through the skin of the legs and are commonly referred to as “spider-veins”. As the disease progresses, the stages become easily visible as varicosities, bulging through the skin in tortuous paths along and around the leg. Pain is felt along the legs and the disease progresses to a third stage where induration and discoloration of the gait area of the lower leg, right above the ankle appear, the so called “ankle flare”. Thinning of the dermis ensues associated with poor blood supply that makes the skin very susceptible to trauma. The smallest scratch will rupture the skin that has little normal blood flow, and the rupture becomes an ulcer that is unsightly, ill-smelling, painful and difficult to heal. Venous ulcers are notoriously slow to heal; one study showed that 50% of ulcers had been open for one year or more. An ulcer may heal by various applications of unguents and salves, bandaging and repeated cleaning, thus reverting to the third stage, but it can also progress and give rise to worsening conditions that may necessitate amputation of the limb. It has been determined that there are approximately 2.6 million venous stasis ulcers that require treatment in the USA yearly. Also it is estimated that all this has as root cause, the dysfunction or destruction of one or more of the valves along the veins of the leg. As such, most treatments to date address the symptoms, not the root cause, of the disease.

In another region of the venous system, the pulmonic or pulmonary valve, external to the heart and carrying venous blood in the direction of the lung, may also be found to be dysfunctional, deformed, or in congenital errors such as pulmonary atresia, be absent and in truth represents another form of venous insufficiency. This condition if not corrected can be fatal. It is thus necessary to find a replacement venous valve (a venous valve device) that will also maintain venous flow in the forward direction.

The valves of the veins in the leg are particularly important because hydrostatic forces encourage retrograde flow in the erect position. Retrograde flow may be permitted in the deep veins because of an absent valve, a vein valve prolapse (floppy valve cusps), valve agger or ring dilatation and fixed cusps (a cusp filled with thrombus) or thickened cusps. Patients present to physicians in different disciplines that treat the symptoms, swelling, pain, venous claudication and cramping, and the skin changes and ulcers, by different methods. In the long run, as explained, the root cause of the disease, the dysfunctional valve, its repair or replacement is the only real remedy. The great majority of disciplines treat CVI conservatively. Vascular surgeons have attempted leaflet repairs and performed a series of axillary valved vein transfers to veins in the leg. These procedures result in some amelioration of symptoms, but recurrence is seen often. Phipher et al (Am J Surg. 1989 June; 157(6):588-592, and Invest Radiol. 1985 January-February; 20(1):42-44) studied biological cardiac replacement valves function in the vena cava of dogs without anticoagulation as a first possible substitute for failed venous valves. In this setting at least 30% of the xenografts performed well. Taheri (Am J Surg. 1988 August; 156(2):111-114. and Int Angiol. 1989 January-March; 8(1):7-9) used mechanical valves made of platinum in the inferior vena cava of mongrel dogs that remained patent for more than 12 months. The same experience was not proved successful in the human.

Quijano et al. in U.S. Pat. No. 5,824,061 and U.S. Pat. No. 7,159,593, entire contents of which are incorporated herein by reference, teach of the use of bovine, equine, (and in general from any quadruped) jugular veins containing integral bileaflet or trileaflet venous valves, preserved by diverse means, but mostly using buffered glutaraldehyde as substitutes for failed venous valves. The patents also teach that the device could also be used as a pulmonic valved conduit for the reconstruction of the right ventricular outflow tract when the pulmonic valve is damaged, absent or dysfunctional. In the past, polymer or plastic materials have been studied. Prosthetic venous valve replacements made of Gore-Tex® PTFE, polyurethane materials have been tried but unfortunately results invariably were suboptimal with thrombus developed quickly, leaflets hardened and incompetence and regurgitation returned. The infection rate of these materials was also subject of great concern.

Gomez-Jorge and Venbrux (J Vasc Interv Radiol. 2000 July-August; 11(7):931-936) used the bovine jugular vein that is placed within a Nitinol commercially available biliary stent and implanted by means of a sheath into the iliac veins of swine. The feasibility of placing a functioning venous valve bioprosthesis in the venous system was demonstrated.

Lane in U.S. Pat. No. 4,904,254, entire contents of which are incorporated herein by reference, teaches a cuff for restoring competence to an incompetent venous valve, the cuff comprising a band of biocompatible implantable material, the band being of sufficient length to encompass the vein at the site of the venous valve with portions of the band overlapping, the overlapping portions being joinable together to form a cuff of desired circumference small enough to restore competence.

Camilli in U.S. Pat. No. 5,358,518, entire contents of which are incorporated herein by reference, discloses an artificial venous valve for insertion into the human venous system, comprising a hollow elongated support and a plate carried by and within the hollow elongated support and movable relative to the support between a position to permit flow of blood in one direction and a position in which to prevent flow of blood in an opposite direction through the support. The plate is movable to open and close over a pressure differential range on opposite sides of the plate of 1-50 mm Hg.

Shaolian et al. in U.S. Pat. No. 6,299,637, entire contents of which are incorporated herein by reference, discloses a self expandable prosthetic venous valve, comprising: a tubular wire support, expandable from a first, reduced diameter to a second enlarged diameter, and having a flow path therethrough; and at least one leaflet pivotably positioned in the flow path for permitting flow in a forward direction and resisting flow in a reverse direction, the leaflet comprising an internal support having a first pivot point and a second pivot point attached to opposing sides of the tubular support, and a rotational axis extending through the first and second pivot points.

Strecker in U.S. Pat. No. 6,602,286, entire contents of which are incorporated herein by reference, discloses a body lumen valve comprising a base, a valve element comprising tissue disposed on a mesh, the valve element connected to the base such that the valve moves relative to the base between an open position and a closed position, and a connector that attaches the base directly to a body lumen surface region.

Crosslinking of biological tissue material is often desired for biomedical or medical device applications. For example, the structural framework of xenogeneic pericardial tissue has been extensively used for manufacturing replacement heart valve bioprostheses and other implanted structures, wherein it provides good biocompatibility and strength. However, biomaterials derived from xenogeneic collagenous tissue must be chemically modified and subsequently sterilized before they can be implanted in humans. The fixation, or crosslinking, of collagenous tissue may increase strength and reduces antigenicity and immunogenicity.

For clinical purposes, fixation of biological tissue is used to reduce antigenicity and immunogenicity and preserve strength, increase durability by prevention of enzymatic degradation. Various crosslinking agents have been used for fixation of biological tissue. The tissue preserving and crosslinking agents used to date, have brought on various serious adverse events to the health of the patients during their use. The most used fixative and sterilant, glutaraldehyde, has provided excellent preservation of xenogeneic collagen, however, depending on the process conditions used, the residuals present in the tissue even after extensive rinsing prior to implantation, present still undesirable toxicity levels, are irritant to human live tissue, and can induce thrombus formation (clots), hemolysis, and fibrin and protein deposition on the tissue implanted, often precipitating the failure of the device. It is therefore desirable to provide a crosslinking agent suitable for use in biomedical applications that will provide acceptable cytotoxicity, absent or decreased irritant effect to the patients' live tissue and that forms stable and biocompatible crosslinked tissue products.

The decellularized pericardial tissue of the present invention is useful in a venous valve device. The segment of pericardial tissue may be in a form of sheet, patch or strip. Forming appropriate segments of tissue sheet are critical in the process of tissue sheet preparation, particularly the free margin or coapting edge of the valve. The cut tissue edge should derive only minimal effect from any cutting energy applied onto the collagenous tissue. A process for forming segments of crosslinked decellularized pericardial tissue is provided. Furthermore, it is disclosed that a process of manufacturing a venous valve device using a shape-memory material wire-frame onto which segments of crosslinked decellularized pericardial tissue sheet were mounted.

### SUMMARY OF THE INVENTION

In general, it is an object of the present invention to provide an implantable venous valve that may be introduced into the defective venous system by minimally traumatic and flow disturbing methods. Thus, a tissue (biological or artificial) construct that closely approximates the shape and that will closely result in function like a natural venous valve is desired. Geometrically therefore, the construct should approximate a venous valve. Measurements of venous valves from bovine, equine, porcine and human origin yield very specific parameters that can then be incorporated into the design of the invention. The native venous valves, undisturbed or fresh from the slaughterhouse, and also in a preserved state by fixation with agents such as glutaraldehyde or the presently proposed preservation methods, were dissected along the longitudinal axis, the vein valve, sinuses, aggers, exposed and their dimensions closely measured.

Translation of their geometrical patterns to drawing then allowed the initiation of formation of the geometrical design of the tissue construct. Initial approximation of agger and leaflet inferior margin of attachment with mathematical functions such as a parabola resulted in shapes that were generally close to the native valves but the ratio of height to width showed some discrepancy. It appeared that the values obtained by iteration of the equation of a catenary yielded an exact likeness of the shape of the margin of attachment, and sinuses of a bovine, equine, porcine and human venous valve. Such equations are shown below:

$$\begin{matrix}y = {x}^{\frac{2}{B}} the parabola ; and B = 1 , 2 , 3 , 4 , … & ( Equation 1 ) \\ y = a cosh ( \frac{x}{a} ) catenary , \\ where a = 2 , 2.1 , 2.2 , 2.3 , 2.4 , … 3.0 & ( Equation 2 )\end{matrix}$$

The geometry of the free margin or coapting edge of the valve can be approximated with a similar catenary equation. Thus, in this manner the shape of the venous valve is defined and experiments suggest that the valve meets the desired specifications in flow control, allowing quasi-laminar flow to pass through the valve, minimizing turbulence that is deleterious and leads to thrombus formation, and providing ample coaptation to ensure the competency of the valve under conditions of diameter changes in the agger or better described as dilatation of the “annulus” of the venous valve.

It is one object of the present invention to provide a graft sheet material for use in a venous valve device, wherein the graft sheet material is formed from a segment of connective tissue protein or collagen, and the segment is decellularized and crosslinked with a crosslinking agent resulting in reasonably acceptable cytotoxicity and reduced enzymatic degradation.

In some aspects, there is provided a biological tissue material or tissue sheet material comprising a process of removing cellular material and lipid from a natural tissue and crosslinking the natural tissue with a crosslinking agent or with ultraviolet irradiation, the tissue material being characterized by reduced antigenicity, reduced immunogenicity and reduced enzymatic degradation upon placement inside or on a patient's body, wherein porosity of the natural tissue is optionally increased. In one aspect, the increase of porosity is adapted for promoting tissue regeneration, when in need. In a preferred embodiment, the natural tissue or tissue sheet material is selected from a group consisting of bovine pericardium, equine pericardium, porcine pericardium, ovine pericardium, caprine pericardium, kangaroo pericardium, fascia lata, dura mater and the like. In still another embodiment, the crosslinked decellularized natural tissue material is loaded with at least one growth factor, at least one bioactive agent, or stem cells/regenerative cells.

In a further embodiment, the tissue sheet material is selected from a group consisting of a bovine pericardium, an equine pericardium, an ovine pericardium, a porcine pericardium, a caprine pericardium, a kangaroo pericardium, fascia lata, dura mater and the like. In another embodiment, the tissue sheet material is crosslinked with a crosslinking agent or with ultraviolet irradiation, wherein the crosslinking agent may be selected from the group consisting of genipin, its analog, derivatives, and combination thereof, epoxy compounds, dialdehyde starch, glutaraldehyde, formaldehyde, dimethyl suberimidate, carbodiimides, succinimidyls, diisocyanates, acyl azide, and combinations thereof. The epoxy compounds are chemically similar structure, generally defined as compounds in which an oxygen atom is directly attached to two adjacent or non-adjacent carbon atoms of a carbon chain or ring system; thus cyclic ethers.

Some aspects of the invention provide a process for the production of a decellularized tissue or tissue sheet, comprising: providing a tissue having cells and extracellular matrix; subjecting the tissue to a solution containing bile acid or bile salts which effect the solubilization of cell membranes of the cells present in the tissue; removing the solubilized cell membranes by flushing the tissue with filtered water; and treating the tissue with a crosslinking agent, such as epoxy compounds.

The process for the production of a decellularized tissue or tissue sheet may further comprise dehydrating the decellularized tissue. Alternately, the dehydrating is carried out by soaking the decellularized tissue in glycerol or in glycerol-alcohol mixture (for example, 80% glycerol-20% ethanol). Alternately, the process may further comprise lyophilizing (freeze-drying) the decellularized tissue or tissue patch/sheet in a sterile environment, preferably removing all or substantial amount of the crosslinking agent. Thus, for its use, a reconstitution with specially formulated solutions or simple sterile de-ionized water or saline may suffice to return the material to its flexible, durable, strong, viable state.

The properties of a vein of a mammal are different from those of an artery. The flow and pressure inside a vein are quite lower than in an artery. Therefore, the structure requirement for a venous vein device is distinctly different from that of a heart valve. Some aspects of the invention provide an implantable venous device made of a sheet of biocompatible material, the device comprising: a longitudinal wire-frame structure having a base, wherein the wire-frame structure is a continuous seamless wire loop, the base of the wire-frame structure being radially inwardly compressible (without soldering, welding, or re-joining); and plural anchoring mesh (lattice) wing members spaced apart and connected to the base of the wire-frame structure, wherein each mesh lattice wing member and the wire-frame are integral parts from the sheet of biocompatible material (without gluing, soldering, welding, or any re-joining operation).

Some aspects of the invention provide an implantable venous valve device made of a sheet of biocompatible material, the device comprising: a longitudinal wire-frame structure having a base, wherein the wire-frame structure is a continuous seamless wire loop, the base of the wire-frame structure being radially inwardly compressible (without soldering, welding, or re-joining), wherein the wire-frame structure is mounted with at least one leaflet to provide a unidirectional fluid flow; and plural anchoring mesh or lattice wing members spaced apart and connected to the base of the wire-frame structure, wherein each mesh lattice wing member and the wire-frame are integral parts from the sheet of biocompatible material (without gluing, soldering, welding, or any re-joining operation).

In one embodiment, the leaflet of the implantable venous valve device is made of a polymer membrane, for example, a polyurethane membrane.

In one embodiment, the leaflet of the implantable venous valve device is a tissue leaflet.

In one embodiment, the leaflet of the implantable venous valve device is a decellularized tissue leaflet.

In one embodiment, the leaflet of the implantable venous valve device is a crosslinked tissue leaflet.

In one embodiment, the leaflet of the implantable venous valve device is a crosslinked tissue leaflet, the leaflet being crosslinked with a crosslinking agent of epoxy compounds.

In one embodiment, the leaflet of the implantable venous valve device is a crosslinked pericardium tissue leaflet.

In one embodiment, the leaflet of the implantable venous valve device is made of a crosslinked pericardium, wherein the pericardium is selected from a group consisting of bovine pericardium, equine pericardium, porcine pericardium, ovine pericardium, caprine pericardium, and kangaroo pericardium.

In one embodiment, the leaflet of the implantable venous valve device is a tissue leaflet made by a process comprising steps of: starting from a tissue sheet having cells and extracellular matrix; subjecting the sheet to a solution containing formulations of bile acids or bile salts that effect the solubilization of cell membranes of the cells present in the tissue sheet; removing the solubilized cell membranes by flushing the tissue sheet with filtered water or saline; and treating the tissue sheet with a crosslinking agent. In an exemplary embodiment, the bile acid is cholic acid or deoxycholic acid. In another exemplary embodiment, the bile salts are glycocholate or deoxycholate.

In one embodiment, the leaflet of the implantable venous valve device is a tissue leaflet made by a process comprising steps of: providing a tissue sheet having cells and extracellular matrix; subjecting the sheet to a solution containing bile acid or bile salts that effect the solubilization of cell membranes of the cells present in the tissue sheet; removing the solubilized cell membranes by flushing the tissue sheet with filtered water or saline; treating the tissue sheet with a crosslinking agent; and dehydrating the decellularized tissue.

In one embodiment, the leaflet of the implantable venous valve device is a tissue leaflet made by a process comprising steps of: providing a tissue sheet having cells and extracellular matrix; subjecting the sheet to a solution containing bile acid or bile salts that effect the solubilization of cell membranes of the cells present in the tissue sheet; removing the solubilized cell membranes by flushing the tissue sheet with filtered water or saline; treating the tissue sheet with a crosslinking agent; and soaking the decellularized tissue in glycerol or glycerol-alcohol mixture.

In one embodiment, the leaflet of the implantable venous valve device is a tissue leaflet made by a process comprising steps of: providing a tissue sheet having cells and extracellular matrix; subjecting the sheet to a solution containing bile acid or bile salts that effect the solubilization of cell membranes of the cells present in the tissue sheet; removing the solubilized cell membranes by flushing the tissue sheet with filtered water or saline; treating the tissue sheet with a crosslinking agent; and lyophilizing the decellularized tissue.

Some aspects of the invention provide an implantable venous filter device made of a sheet of biocompatible material, the device comprising: a longitudinal wire-frame structure having a base, wherein the wire-frame structure is a continuous seamless wire loop, the base of the wire-frame structure being radially inwardly compressible, wherein the wire-frame structure comprises a filter mechanism; and plural anchoring mesh (lattice) wing members spaced apart and connected to the base of the wire-frame structure, wherein each mesh lattice wing member and the wire-frame are integral parts from the same sheet of biocompatible material.

Some aspects of the invention provide an implantable venous valve device made of a sheet of biocompatible material, the device comprising: a longitudinal wire-frame structure having a base, wherein the wire-frame structure is a continuous seamless wire loop, wherein the wire-frame structure is made of a shape memory Nitinol alloy, the base of the wire-frame structure being radially inwardly compressible; and plural anchoring mesh (lattice) wing members spaced apart and connected to the base of the wire-frame structure, wherein each mesh lattice wing member and the wire-frame are integral parts from the sheet of biocompatible material (without gluing, soldering, welding, or any re-joining operation). In one embodiment, the wire-frame structure is mounted with at least one leaflet to provide a unidirectional fluid flow when located in a flow stream.

Some aspects of the invention provide an implantable venous valve device made of a sheet of biocompatible material, the device comprising: a longitudinal wire-frame structure with a base, wherein the wire-frame structure is a continuous seamless wire loop, wherein the wire-frame structure is radially inwardly compressible, the base of the wire-frame structure being radially inwardly compressible; and plural anchoring mesh (lattice) wing members spaced apart and connected to the base of the wire-frame structure, wherein each mesh lattice wing member and the wire-frame are integral parts from the sheet of biocompatible material (without gluing, soldering, welding, or any re-joining operation), the anchoring mesh lattice wing members being neither radially inwardly compressible nor outwardly expansible. In one embodiment, the wire-frame structure is mounted with at least one leaflet to provide a unidirectional fluid flow.

Some aspects of the invention provide an implantable venous valve device made of a sheet of biocompatible material, the device comprising: a longitudinal wire-frame structure with a base, wherein the wire-frame structure is a continuous seamless wire loop, wherein the wire-frame structure is radially self-expandable, the base of the wire-frame structure being radially inwardly compressible; and plural anchoring mesh (lattice) wing members spaced apart and connected to the base of the wire-frame structure, wherein each mesh lattice wing member and the wire-frame are integral parts from the sheet of biocompatible material (without gluing, soldering, welding, or any re-joining operation). In one embodiment, the wire-frame structure is mounted with at least one leaflet to provide a unidirectional fluid flow.

Some aspects of the invention provide a process of manufacturing the venous device comprising: (a) providing the sheet of biocompatible material; (b) laser-cutting the sheet to form the plurality of mesh lattice wing members and a central wire member that connects the plural mesh lattice wing members on a plane, wherein the central wire member has at least two wire sections being detached from the plural mesh lattice wing member; (c) forming an individual wire-frame configuration on each of the at least two wire sections by pushing upward a central part of each wire section while holding the mesh lattice wing members on the plane; and (d) bending the mesh lattice wing members to be substantially parallel to a direction of the wire-frame configuration.

### BRIEF DESCRIPTION OF THE DRAWINGS

FIG. 1 shows a schematic illustration of a plotted water knife type (liquid-jet) cutting apparatus for precision cutting of tissue segments.

FIG. 2 shows steps of laser-cutting a sheet material in the manufacturing process of a venous device.

FIG. 3 shows the first step of forming the wire-frame from a laser-cut sheet material in the manufacturing process of a venous device.

FIG. 4 shows a side view of the laser-cut sheet material of FIG. 3, with steps of forming the wire-frame in the manufacturing process of a venous device.

FIG. 5 shows a perspective view of the self-expandable venous device of the present invention, including two anchoring support wing members.

FIG. 6 shows a perspective view of the self-expandable venous valve device of the present invention, including two anchoring support wing members.

FIG. 7 shows a top view, section I-I of FIG. 6, of the self-expandable venous valve device, including two coaptable leaflets.

FIG. 8 shows one embodiment of forming an alternate wire-frame from a laser-cut sheet material in the manufacturing process of a venous device.

FIG. 9 shows the first step of forming a preferred wire-frame from a laser-cut sheet material in the manufacturing process of a venous device.

FIG. 10 shows a perspective view of the self-expandable venous device of the present invention, including three anchoring support wing members.

### DETAILED DESCRIPTION OF EXEMPLARY EMBODIMENTS

The following detailed description is of the best presently contemplated modes of carrying out the invention. This description is not to be taken in a limiting sense, but is made merely for the purposes of illustrating general principles of embodiments of the invention.

A “tissue” or “tissue material” refers to a material of biological tissue origin that might be decellularized and crosslinked to form or used in a medical device. A tissue sheet, such as a pericardial sheet, is in a sub-group of tissue material (including sheet form and non-sheet form).

An “implant” refers to a medical device which is inserted into, or grafted onto, bodily tissue to remain for a period of time, such as an extended-release drug delivery device, tissue valve, replacement venous valve, drug-eluting stent, vascular graft, wound healing or skin graft, orthopedic prosthesis, such as bone, ligament, tendon, cartilage, and muscle.

A “decellularization process” is meant to indicate the process for detaching and removing a substantial portion or all of cellular substance from cellular tissue and/or tissue matrix that contains connective tissue protein/collagen, for example, a pericardial sheet.

It is one object of the present invention to provide a decellularized biological scaffold chemically treated with a crosslinking agent that may be configured and adapted for tissue regeneration/tissue engineering or other surgical/medical applications with no regeneration. In the region having suitable substrate diffusivity, a decellularized biological tissue material with added porosity and chemically treated by a crosslinking agent enables biodurability and/or tissue engineering in many biomedical applications.

Cell Membranes

Every cell is surrounded by a plasma membrane that creates a compartment where the functions of life can proceed in relative isolation from the outside world. Biological membranes consist primarily of protein and lipids; for example, the myelin sheath membrane consists of about 80% lipid and 20% protein. Two main types of lipids occur in biological membranes: phospholipids and sterols. The bile salts are critically important for the solubilization of lipids in a body. For example, it is known that bile salts emulsify fats in the intestine. The hydrophobic side or surface of the bile salt associates with triacylglycerols to form a complex. These complexes aggregate to form a micelle, with the hydrophilic side of the bile salt facing outward. The micelles (that detached from the surface of the extracellular matrix inside the tissue or tissue sheet) would be relatively easy to remove from the extracellular space in the decellularization process.

There are currently two mechanisms for tissue sheet or tissue material decellularization. The conventional decellularization process is to increase the differential osmotic pressure across the cellular membrane until the membrane ruptures. It is usually achieved by exposing the cells to a fluid with lower osmotic pressure, for example deionized water, via a reverse osmosis process. This approach leaves substantial cellular residues or material within the extracellular space still attached/connected to certain internal surface of the tissue sheet and these residues may have deleterious effects on the survival of the implant. On the contrary, the decellularization approach of the present invention is to delipid or to solubilize lipids (such as the lipids of the membranes), instead of merely breaking up the membranes.

The decellularized pericardial sheet would contain less cellular residues because the solubilized membrane detaches from the surface of certain extracellular matrix inside the tissue sheet and is relatively easy to remove since it is already dissociated/detached and free to move around. The majority of the cellular residues having solubilized lipids are much easier to be removed from the extracellular space, for example, by rinsing or flushing with filtered water, sterile saline, sterile alcohol solution or other appropriate solvents.

In co-pending application Ser. No. 11/704,645, filed Feb. 9, 2007 and Ser. No. 11/704,563, filed Feb. 9, 2007, it is disclosed a process for manufacturing a pericardial tissue sheet of the present invention having main steps of cleaning, bioburden reduction, decellularization, crosslinking, and sterilization, with optional steps of porosity enhancing, lyophilization, and glycerol soaking.

Cholic Acid in Decellularization Process

Cholic acid, shown below, has an empirical formula of C₂₄H₄₀O₅.

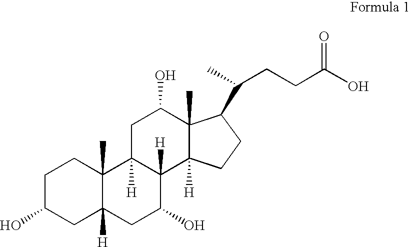

Cholic acid is a bile acid, a white crystalline substance insoluble in water, with a melting point of 200-201° C. Salts of cholic acid (also broadly herein including derivatives of cholic acid) are called cholates or bile salts. Cholic acid is one of the four main acids produced by the liver where it is synthesized from cholesterol. It has active side groups (COOH and OH) and is soluble in alcohol and acetic acid. Cholic acid possesses a particular hydrogen (the singular ‘H’ shown at the left lower corner of the structure formula 1 above). As a result, the first six-carbon ring on its right-hand side and the second six-carbon ring on its left-hand side are no longer coplanar but have a cis-configuration (a three-dimension structure). This cis-configuration of two contiguous six-carbon rings improves the detergent properties of the bile acids so they are better able to solubilize lipids.

Glycocholate is an example of a bile salt, derived from glycocholate acid as shown below:

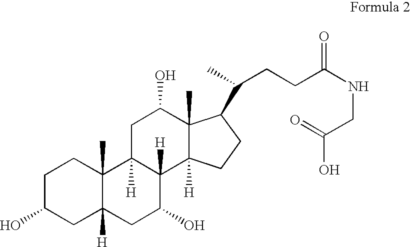

The cholic acid forms a conjugate with taurine, yielding taurocholic acid. Cholic acid and chenodeoxycholic acid are the most important human bile acids. Some other mammals synthesize predominantly deoxycholic acid. The main use of cholic acid is as an intermediate for the production of ursodeoxycholic acid. Ursodeoxycholic acid is a pharmaceutical product that is used for several indications including the dissolution of gallstones and the treatment and prevention of liver disease. Cholic acid (broadly herein defined to include its derivatives) has many different uses in traditional Chinese medicine. Its main use is as an ingredient in the manufacture of artificial calculus bovis (artificial gallstones).

Deoxycholic acid with an empirical formula of C₂₄H₄₀O₄, is shown below:

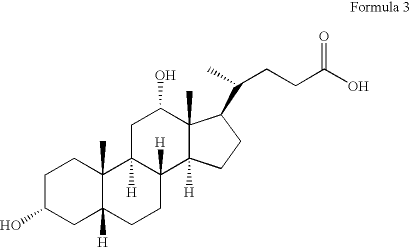

Deoxycholic acid is sparingly soluble in water, but soluble in alcohol and to a lesser extent acetone and glacial acetic acid. Historically, deoxycholic acid was used as an intermediate for the production of corticosteroids, which have anti-inflammatory indications.

An emerging use of deoxycholic acid is as a biological detergent to lyse cells and solubilize cellular and membrane components. Some aspects of the invention relate to a process of decellularization of tissue or tissue biomaterial via delipidation as a medical device. It is stipulated that cell extraction resulting from cholic acid decellularization removes lipid membranes and membrane-associated antigens as well as soluble proteins (since cell membranes have been dissolved). In one embodiment, the process of delipidation or decellularization via delipidation of tissue or tissue biomaterial utilizes cholic acid, deoxycholic acid, or bile salts (including salts of cholic acid and its derivatives, such as glycocholate and deoxycholate) sufficient to delipid and subsequently decellularize the tissue biomaterial.

In a preferred embodiment, the delipidated and/or decellularized tissue or tissue biomaterial is further crosslinked (for example, through ultraviolet irradiation) or treated with a chemical agent, such as genipin, its analog, derivatives, and combination thereof, epoxy compounds, dialdehyde starch, glutaraldehyde, formaldehyde, dimethyl suberimidate, carbodiimides, succinimidyls, diisocyanates, acyl azide, and combinations thereof. Other crosslinking means may also be applicable to crosslink the decellularized tissue (pericardial and non-pericardial tissues) of the present invention.

Girardot in U.S. Pat. No. 4,976,733, entire contents of which are incorporated herein by reference, discloses a prosthesis having an amount of an anticalcification agent covalently coupled thereto, which anticalcification agent comprises an aliphatic straight-chain or branched-chain, saturated or unsaturated, carboxylic acid or a derivative thereof, which acid contains from about 8 to about 30 carbon atoms, and which acid is substituted with an amino group, a mercapto group, a carboxyl group or a hydroxyl group, which group is covalently coupled to the prosthesis. In one preferred embodiment, the delipidated and/or decellularized tissue or tissue biomaterial is further treated with the above-cited anticalcification agent.

Cholic acid and deoxycholic acid has a low acute toxicity, with LD₅₀ i.v. 50 mg/kg and 15 mg/kg in rabbit, respectively. In general, bile acids and salts have only a minor toxic potential when given by mouth. In large doses, they are likely to have the same effects as saponins; the main action is likely to be irritation of mucous membranes. Parenterally they are much more toxic and may cause hemolysis, a digitalis-like action on the heart and effects on the central nervous system.

Bile is a bitter, yellow to greenish fluid composed of glycine or taurine conjugated bile salts, cholesterol, phospholipid, bilirubin diglucuronide, and electrolytes. It is secreted by the liver and delivered to the duodenum to aid the process of digestion and fat absorption by emulsification of fat products in the upper small intestine. They play role of dissolving cholesterol and accretes into lumps in the gall bladder, forming gallstones. Bile's bicarbonate constituent serves for alkalinizing the intestinal contents. Bile is responsible for as the route of excretion for hemoglobin breakdown products (bilirubin). Excretion of bile salts by liver cells and secretion of bicarbonate rich fluid by ductular cells in response to secretion are the major factors that normally determine the volume of secretion. Bile acids are liver-generated steroid carboxylic acids. Examples of bile acids include cholic acid itself, deoxycholic acid, chenodeoxy colic acid, lithocholic acid, taurodeoxycholate ursodeoxycholic acid, hyodeoxycholic acid and derivatives like glyco-, tauro-, amidopropyl-1-propanesulfonic- and amidopropyl-2-hydroxy-1-propanesulfonic-derivatives of the above bile acids, or N,N-bis(3D Gluconoamidopropyl) deoxycholamide. Salts of bile acids are normally called bile salts.

The primary bile acids (for example, cholic and chenodeoxycholic acid) are conjugated with either glycine or taurine in the form of taurocholic acid and glycocholic acid. The secondary bile acids (deoxycholic, lithocholic, and ursodeoxycholic acid) are formed from the primary bile acids by the action of intestinal bacteria. They are soluble in alcohol and acetic acid. The lithocolyl conjugates are relatively insoluble; excreted mostly in the form of sulfate esters like sulfolithocholylglycine. Most of the bile acids are reabsorbed and returned to the liver via enterohepatic circulation, where, after free acids are reconjugated, they are again excreted.

Tissue Specimen Preparation

In one embodiment of the present invention, porcine pericardia procured from a slaughterhouse are used as raw material. In the laboratory, the pericardia are first gently rinsed with fresh saline to remove excess blood on tissue. The cleaned pericardium before delipidation process is herein coded specimen-A. The procedure used to delipid the porcine pericardia is described below: A portion of the trimmed pericardia is immersed in a hypotonic tris buffer (pH 8.0) containing a protease inhibitor (phenylmethyl-sulfonyl fluoride, 0.35 mg/L) for 24 hours at 4° C. under constant stirring. Subsequently, they are immersed in a 1% solution of Triton X-100 (octylphenoxypolyethoxyethanol; Sigma Chemical, St. Louis, Mo., USA) in tris-buffered salt solution with protease inhibition for 24 hours at 4° C. under constant stirring.

Samples then are thoroughly rinsed in Hanks' physiological solution and treated with a diluted cholic acid about 5% at 37° C. for 1 hour. In one embodiment, the cholic acid solution could be from about 1% to about 99%, preferably about 5% to about 50%. The treatment temperature could be in the range of about 20° C. to 45° C. The treatment period could be from several minutes to 24 hours. This is followed by a further 24-hour extraction with Triton X-100 in tris buffer. This step of the decellularization via cholic acid treatment is to delipid or to solubilize lipids (such as the lipids of the cell membranes), instead of merely breaking up the cell membranes mechanically. The decellularized pericardial sheet would contain less cellular residues because the solubilized membrane detaches from the surface of the extracellular matrix inside the tissue sheet and is relatively easy to remove since it is already partially dissociated/detached and free to move around. Finally, all samples are washed for 48 hours in Hanks' solution and the decellularized sample is coded specimen-B. The majority of the cellular residues having solubilized lipids are much easier to be removed from the extracellular space, for example, by rinsing or flushing with filtered water, sterile saline, sterile alcohol solution or other appropriate solvents. Light microscopic examination of histological sections from the treated tissue of the present invention revealed an intact connective tissue matrix with no evidence of cells or cellular residues.

A portion of the decellularized tissue of porcine pericardia (specimen-B) is thereafter lyophilized at about −50° C. for 24 hours, followed by soaking in glycerol-containing fluid (e.g., 75% glycerol and 25% ethanol) to obtain the decellularized dehydrated pericardia. In other experiments, the glycerol content of the glycerol-alcohol mixture may range from about 50 to 100%. In another example, a portion of specimen-B is rinsed and soaked in glycerol-containing fluid (e.g., 80% glycerol and 20% ethanol) to yield decellularized “dry” dehydrated pericardia; optionally, the decellularized dehydrated pericardium is lyophilized at about −50° C. for 24 hours to get a substantially “moisture-free” dehydrated decellularized pericardium. The dehydrated decellularized tissue or pericardial tissue can be re-constituted for medical applications. In a preferred embodiment, the decellularized tissue before lyophilization is thoroughly flushed to remove crosslinking agent (for example, epoxy compounds), In another preferred embodiment, the decellularized tissue before lyophilization is treated with a counter-reactive agent (i.e., neutralizing agent) for a particular crosslinking agent; for example, an amine-containing compound is used to react with the excess free crosslinking agent of epoxy compounds and therefore, deactivate the excess crosslinking agent remained in the tissue. Other lyophilization conditions may also apply, such as between −50° C. and −10° C.

Tissue Specimen Crosslinking

The decellularized tissue (specimen-B) of porcine pericardia are fixed with various crosslinking agents. The first specimen is fixed in 0.625% aqueous glutaraldehyde (Merck KGaA, Darmstadt, Germany) as reference. The second specimen is fixed in genipin (Challenge Bioproducts, Taiwan) solution at 37° C. for 3 days. The third specimen is fixed in 4% epoxy solution (ethylene glycol diglycidyl ether) at 37° C. for 3 days. The chemical structure for ethylene glycol diglycidyl ether, one exemplary epoxy compound cited herein, is shown below:

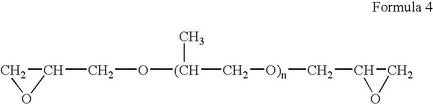

The aqueous glutaraldehyde, and genipin used are buffered with phosphate buffered saline (PBS, 0.01M, pH 7.4). The aqueous epoxy solution was buffered with sodium carbonate/sodium bicarbonate (0.21M/0.02M, pH 10.5). Different buffer solution systems are used for controlling at each desired reactive pH buffer range. The amount of solution used in each fixation was approximately 200 mL for a 10 cm×10 cm porcine pericardium. Subsequently, the fixed decellularized specimens are sterilized in a graded series of ethanol solutions with a gradual increase in concentration from 20 to 75% over a period of 4 hours. Finally, the specimens are thoroughly rinsed in sterilized PBS for approximately 1 day, with solution change several times (2 to 6 times), and prepared for tissue characterization with respect to degree of crosslinking and appearance. All specimens show crosslinking characteristics per analysis of amino acid residue reactions, increased denaturation temperatures, and resistance against collagenase degradation. The epoxy compounds crosslinked specimen shows whitish translucent appearance with soft flexible feeling (to be used as venous valve leaflets later); the glutaraldehyde crosslinked specimen shows yellowish appearance with semi-rigid feeling; and the genipin crosslinked specimen shows dark bluish appearance with flexible feeling.

Though certain methods for removing cells from cellular are well known to those who are skilled in the art, it is one object of the present invention to provide a decellularized biological scaffold chemically treated with cholic acid or salts of cholic acid (for example, bile salts) as means of decellularization having porosity for future biomedical application. Some aspects of the invention provide a process for the production of a decellularized pericardial tissue (patch, sheet, strip, and other appropriate shapes or configurations) comprising: (a) providing a pericardium tissue sheet having cells and extracellular matrix; (b) subjecting the sheet to a solution containing bile acid or bile salts which effect the solubilization of cell membranes of the cells present in the tissue sheet and detachment of the cells from the extracellular matrix; (c) removing the solubilized cell membranes by flushing the tissue sheet with filtered water or other appropriate solution; and (d) treating the tissue sheet with a crosslinking agent (for example, epoxy compounds). The bile acid may be cholic acid or its derivatives whereas the bile salts may be glycocholate, deoxycholate, or other cholates.

Tissue Segmentation with Liquid-Jet Knife

One aspect of the present invention relates to a method for forming segments of a decellularized crosslinked tissue using a non-contact, little or no energy cutting means, such as a focused high-pressure liquid-jet knife. Instead of using a scalpel or laser to cut and remove tissue, the SpineJet® System (manufactured by Hydrocision, Inc., Billerica, Mass.) uses a high-powered stream of water as a cutting means. U.S. Pat. No. 7,122,017, entire contents of which are incorporated herein by reference, discloses surgical liquid jet instruments having a pressure lumen and an evacuation lumen, where the pressure lumen includes at least one nozzle for forming a liquid jet and where the evacuation lumen includes a jet-receiving opening for receiving the liquid jet when the instrument is in operation. In some embodiments, the pressure lumen and the evacuation lumen of the surgical liquid jet instruments are constructed and positionable relative to each other so that the liquid comprising the liquid jet, and any tissue or material entrained by the liquid jet can be evacuated through the evacuation lumen without the need for an external source of suction.

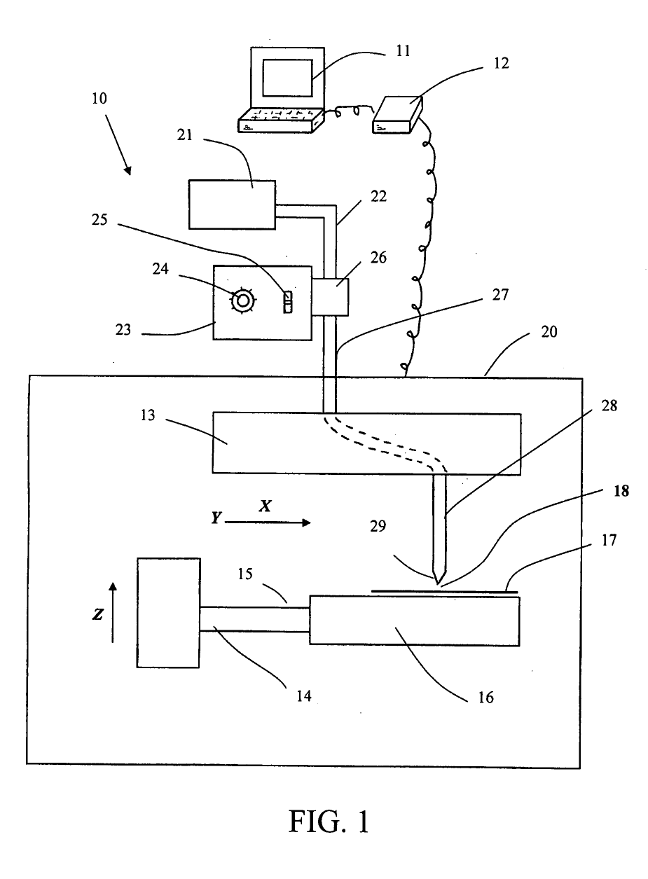

FIG. 1 shows a schematic view of a plotted water knife type (liquid-jet) cutting apparatus for precision cutting of tissue segments. With reference specifically to FIG. 1, the liquid-jet cutting apparatus (10) comprises a liquid-jet system (20) and a computer (11). The liquid-jet system (20) comprises a high-pressure liquid inlet (27), a motion system (13) and a support platform (15). The liquid-jet nozzle (29) is configured to create and direct a focused liquid-jet stream (18) on the support platform (15), which is configured to support the source material (17), such as a tissue sheet or pericardial tissue sheet. The focused liquid-jet (18) is configured to cut through the source material (17) instantaneously in order to cut out a segment according to a prescribed pattern, preferably using a computer controlled software program. The nozzle is preferably arranged not to contact the source material. The tissue sheet or source material of the present invention to be cut may be in a wet stage or moisture-free stage (such as the one containing glycerol as disclosed above), and preferably not immersed in a liquid.

The motion system (13) preferably is arranged to selectively locate and move the position of the focused liquid-jet stream (18) relative to the platform (15) in order to cut the segment out of the source material (17). In the illustrated embodiment, the motion system (13) can move the liquid-jet stream's position along horizontal X-axis and Y-axis. The support platform (15) is vertically movable along a vertical Z-axis. It is to be understood that, in other embodiments, other types of motion systems can be employed.

The computer (11) preferably controls the liquid-jet system (20) via a printer driver (12), which communicates data from the computer (11) to the liquid-jet system (20) in order to control liquid-jet parameters and motion. In the illustrated embodiment, a computer assisted design (CAD) software program is hosted by the computer (11). The CAD software is used to create designs of segments that will be cut. In a preferred embodiment, the CAD software also functions as a command interface for submitting a cutting pattern to the liquid-jet system (20) through the printer driver (12). When directed to do so by the computer (11) and printer driver (12), the liquid-jet system (20) precisely cuts the pattern from the source material (17).

In an alternate embodiment of a liquid-jet cutting apparatus for cutting curved or tubular materials, the support surface (15) comprises a rotary axis (14) configured to accept a tubular or curved source material (16) on the rotary axis. In addition to vertical movement about a Z-axis, the rotary axis (14) is adapted to rotate in order to help position the tubular or curved source material in an advantageous cutting position relative to the focused liquid-jet stream (18).

In a particular embodiment illustrated, the liquid-jet stream (18) is directed perpendicularly with respect to the horizontal X-Y plane. In an alternate embodiment, the focused liquid-jet may be at an angle with respect to the source tissue material (17) on the X-Y plane to have an angled cut. The pressure lumen (28) is preferably constructed from stainless steel, however, in alternative embodiments, the lumen may be constructed from other suitable materials, for example certain polymeric materials, as apparent to those of ordinary skill in the art. Regardless of the specific material from which the pressure lumen is constructed, the pressure lumen must have sufficient burst strength to enable it to conduct a high-pressure liquid to nozzle (29) in order to form the liquid jet (18). The burst strength of the pressure lumen should be selected to meet and preferably exceed the highest contemplated pressure of the liquid supplied for tissue or tissue sheet cutting. Typically, the liquid-jet system (20) will operate at a liquid pressure between about 10 psig and about 10,000 psig, preferably between about 50 psig and about 1,000 psig, depending on the intended material to be cut. Those of ordinary skill in the art will readily be able to select appropriate materials for forming the pressure lumen for particular requirements.

The pressure lumen (28) is in fluid communication with a high-pressure pump (26) via a high-pressure liquid supply conduit (27). The high-pressure liquid supply conduit (27) must also have a burst strength capable of withstanding the highest liquid pressures contemplated for using the apparatus for a particular application. In some embodiments, the high-pressure liquid supply conduit (27) comprises a burst-resistant stainless steel hypotube constructed to withstand at least 10,000 psig. In some embodiments, the hypotube may be helically coiled to improve the flexibility and maneuverability of the liquid-jet apparatus. In preferred embodiments, the high-pressure liquid supply conduit (27) comprises a Kevlar reinforced nylon tube that is connectable to the pressure lumen.

In fluid communication with the high-pressure liquid supply conduit (27) is a high-pressure pump (26), which can be any suitable pump capable of supplying the liquid pressures required for performing the desired procedure. Those of ordinary skill in the art will readily appreciate that many types of high pressure pumps may be utilized for the present purpose, including, but not limited to, piston pumps and diaphragm pumps. In preferred embodiments, the high-pressure pump (26) comprises a disposable piston or diaphragm pump, which is coupled to a reusable pump drive console (23). The high-pressure pump (26) has an inlet that is in fluid communication with a low-pressure liquid supply line (22), which receives liquid from a liquid supply reservoir (21). The pump drive console (23) preferably includes an electric motor that can be utilized to provide a driving force to the high-pressure pump (26) for supplying a high-pressure liquid in liquid supply conduit (27).

In some embodiments, the preferred pump drive console (23) includes a constant speed electric motor that can be turned on and off by means of an operator-controlled switch (25). In some embodiments, the pump drive console (23) can have a delivery pressure/flow rate that is factory preset and not adjustable in use. In other embodiments, the pressure/flow rate may be controlled by the operator via an adjustable pressure/flow rate control component (24) that can control the motor speed of the pump drive console and/or the displacement of the high-pressure pump. In yet other embodiments, the pump drive console (23) and the high-pressure pump (26) may be replaced by a high-pressure liquid dispenser or other means to deliver a high-pressure liquid, as apparent to those of ordinary skill in the art.

The liquid utilized for forming the liquid-cutting jet can be any fluid that can be maintained in a liquid state at the pressures and temperatures contemplated for performing the operations. In some embodiments, in order to improve the cutting character of the liquid jet, the liquid may contain solid abrasives, or the liquid may comprise a liquefied gas, for example carbon dioxide, which forms solid particulate material upon being admitted from the nozzle (29) to form the liquid-jet (18).

Some aspects of the invention provide a process for the production of a decellularized tissue, comprising: (a) providing a tissue sheet having cells and extracellular matrix; (b) treating the tissue sheet with a crosslinking agent; and (c) cutting a segment of tissue out of the tissue sheet with a focused high-pressure liquid-jet, wherein the liquid-jet is supplied at a pressure between about 10 psig and about 10,000 psig, preferably between about 50 psig and about 1,000 psig from a nozzle of the liquid-jet apparatus. The process may further comprise steps of (d) dehydrating the decellularized tissue by soaking the decellularized tissue in glycerol or glycerol-alcohol mixture; and (e) lyophilizing the decellularized tissue. In one embodiment, the segment is sized to form a venous valve leaflet that is appropriately suitable for mounting on a wire-frame of a venous valve device (preferably, a self-expandable venous valve for percutaneous implantation).

In one embodiment, the cross-sectional area of the nozzle is slightly less than that cross-sectional of the pressure lumen. The ratio of the cross-sectional area of the nozzle to that of the pressure lumen may be designed between about 1:2 to about 1:2,000, preferably between about 1:5 to about 1:100. In one preferred embodiment, the liquid-jet is operated in a pulsed manner. In another embodiment, the liquid-jet is operated with a spot size of about 10 μm to 200 μm, preferably about 25 μm to about 100 μm, in diameter at the tissue contact site, thereby producing a cut edge without significantly burning the pericardium adjacent the cut edge.

Self-Expandable Venous Device

Some aspects of the invention provide an implantable venous device made of a sheet of rigid or semi-rigid biocompatible material, the device comprising: a longitudinal wire-frame structure with a base, wherein the wire-frame structure is a continuous seamless wire loop without soldering, welding, or re-joining, the base of the wire-frame structure being radially inwardly compressible; and plural anchoring mesh (lattice) wing members spaced apart and connected to the base of the wire-frame structure, wherein each mesh lattice wing member and the wire-frame are integral parts from the sheet of biocompatible material without gluing, soldering, welding, or any re-joining operation. The longitudinal wire-frame structure is further characterized to be radially inwardly collapsible and outwardly expandable.

In one embodiment, the rigid or semi-rigid biocompatible material may utilize resilient metals, such as a superelastic shape memory alloy, e.g., Nitinol alloys, tempered stainless steel, spring stainless steels, or the like. In some embodiment, the longitudinal wire-frame structure functions as a support to the mounted leaflet (also known as ‘flow stoppage element’) whereas the mesh lattice wing members assume a function of supporting or anchoring the device in place. In another embodiment, the longitudinal wire-frame structure is characterized with a distal commissar and a proximal base with respect to a venous flow in the vein.

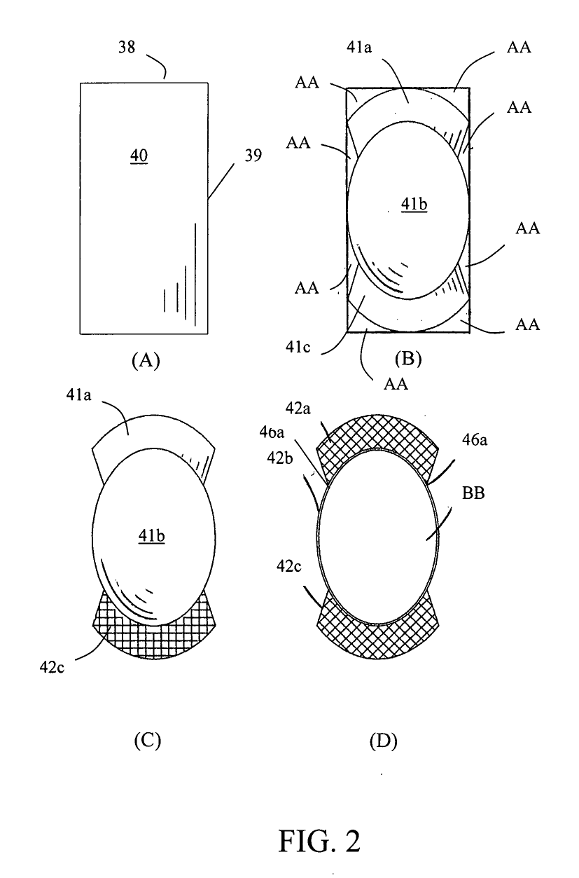

FIG. 2 shows one embodiment of means and steps for laser-cutting a sheet material in the manufacturing process of a supporting structure for a venous device, preferably a self-expandable venous device. The manufacturing process may start with a memory material sheet (40) as shown in FIG. 2A. The memory material may comprise a Nitinol, preferably a temperature sensitive Nitinol. The thickness of the sheet is sized appropriately to provide adequate support for the future venous valve implantation with durability and flexibility. In one embodiment, the starting sheet may be a little curved along the short edgeline (38) with a straight long edgeline (39). In one embodiment, the thickness is in the range of about 0.01 to 1 mm.

By using a laser instrument, a chemical etching process, or a micro-machine (e.g., a computer numeric controlled instrument or machine), the sheet (40) is cut so the area “AA” is cut off and disposed of as shown in FIG. 2B. The remaining sheet contains a first wing member (41a), a second wing member (41c) and a central member (41b) that connects the first and the second wing members. The top view of the central member is about the round shape, slightly oval shape, or a combination of various oval shapes. In one embodiment, the first wing member may be a mirror image of the second wing member. In another embodiment, the two wing members are asymmetric with respect to the central member.

By using a laser instrument or a micro-machine, the second wing member is cut to show mesh, mesh like, scaffold, or stent-like structure with connected struts and is coded as ‘second wing mesh member’ (42c) with bending flexibility and supporting function (shown in FIG. 2C). Similarly, the first wing member may be cut to show mesh, mesh like, scaffold, or stent-like structure with struts and is coded as ‘first wing mesh member’ (42a) with bending flexibility (shown in FIG. 2D). The cross-section profile of the struts is in a rectangular shape, a square shape, a round shape, an oval shape or other appropriate shapes. The cross-section area of the struts is generally in the range of 0.005 to 0.5 mm². The center portion of the central member is similarly cut so the area “BB” is cut off and the central member shows a wire like structure and is coded as ‘central wire member’ (42b).

In one preferred embodiment, the central wire member has a smooth periphery and uniform wire thickness. In one embodiment, the cross-section of the wire of the central wire member is oval or round shape. The cross-section area of the wire of the central wire member is generally in the range of 0.005 to 0.5 mm². The central wire member is inherently connected to the first wing mesh member at least two connecting points (such as 46a), wherein the connection is an integral form from the original one-piece sheet material. In one embodiment, the connecting point is at the intersection of the end mesh (49a or 49b) and the central wire member (42b). Similarly, the central wire member is connected to the second wing mesh member at least two connecting points (such as 46b).

In one embodiment, each mesh strut of the wing mesh members (42a, 42c) comprises a first end and a second end. In some embodiment, the second end of a first mesh strut is connected with a second end of a (diagonal) second mesh strut. In another embodiment, a first end of some mesh struts is connected to the wire of the central wire member. In still another embodiment, a first end of some mesh struts is adjacent to but not connected to the central wire member (shown in FIG. 2C). The term “connecting” is herein intended to explain a phenomenon of ‘a nature extension from a first element to a second element’ without any process of soldering, welding, gluing or re-joining at the intersection.

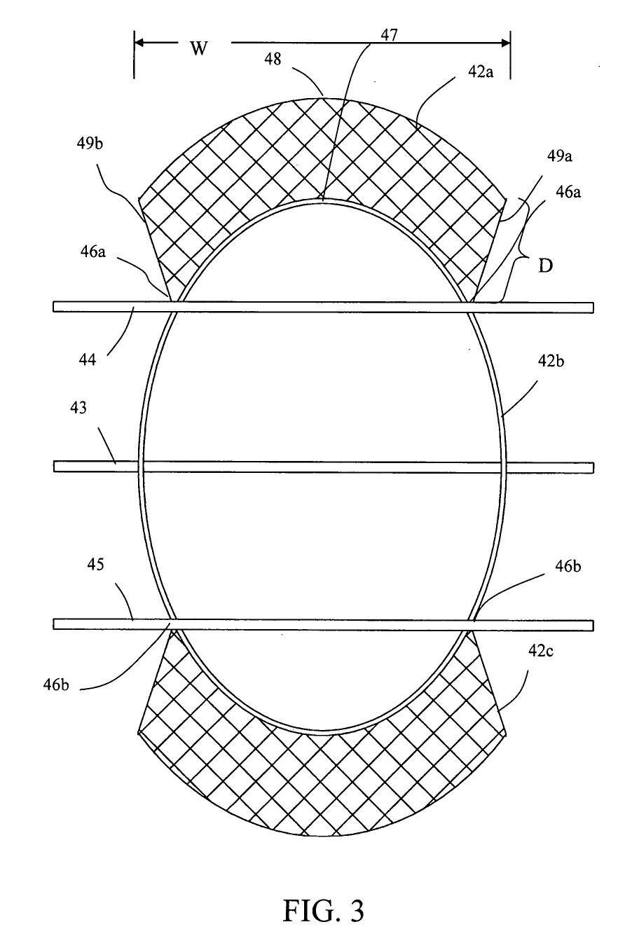

FIG. 3 shows the first step of forming the wire-frame from a laser-cut structure in the manufacturing process of a venous device. Firstly, one primary rigid rod (or stick or bar) (43) is releasably placed at about the middle part of and under the central wire member (42b), as viewed from top of the laser-cut structure. A first auxiliary rigid rod or stick (44) is releasably placed at about the connecting points (46a) and above the central wire member whereas a second auxiliary rigid rod or stick (45) is releasably placed on the opposing side of the central wire member at about the connecting points (46b) and above the central wire member, again viewed from top of the laser-cut structure in FIG. 3. All the rigid rods are relatively rigid (that is, unbendable) as compared to the central wire member during the wire-frame forming steps.

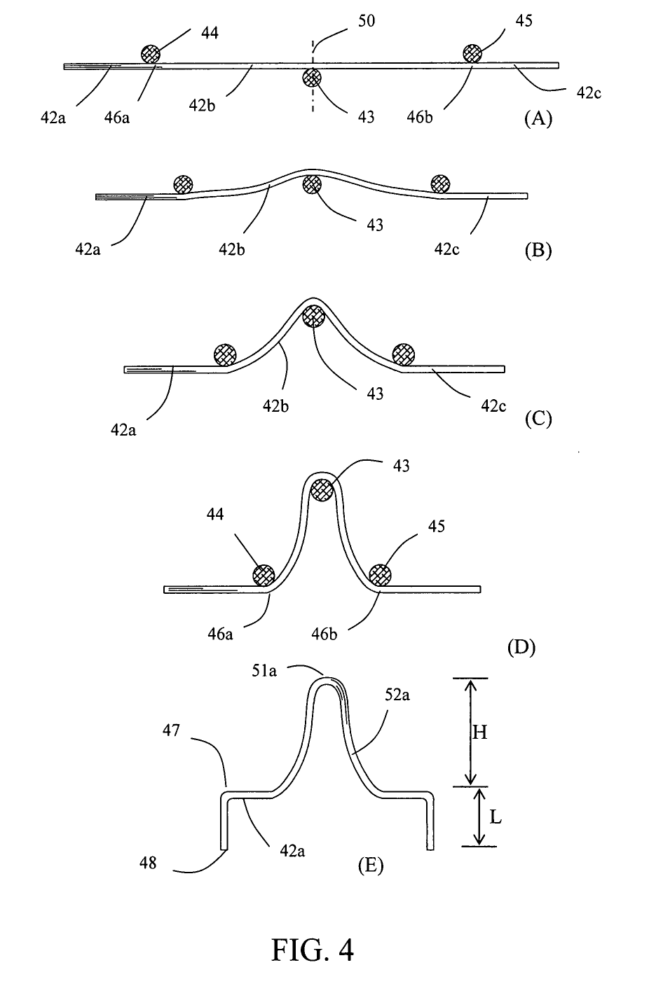

FIG. 4 shows a side view of the laser-cut structure of FIG. 3, with steps of forming the wire-frame in the manufacturing process of a venous device, wherein the series of figures shows a side view of the setup shown in FIG. 3, the view being perpendicular to the axis of the rods (43, 44, and 45). By holding the two auxiliary rigid rods steady, one can push the primary rod (43) upward along the centerline (50) (as shown in FIG. 4A). FIGS. 4B to 4D show a serial process of continuously pushing the primary rod upward and moving the auxiliary rods horizontally towards the centerline.

Finally, a desired height “H” and shape for the commissar (51a and 51b) with a suitable wire-frame structure configuration (52a and 52b) is reached as shown in FIG. 4E. The wire-frame configuration may be further manipulated or processed to provide the optimal structure for mounting the leaflet, for radially collapsing the structure to be inserted in a delivery catheter, or for anchoring the device in a venous vessel after deployment. To make the wire-frame suitable for placement in a tubular venous vessel, at least a portion of the side mesh lattice wing member (42a or 42c) is bent downward at a bending point (47) whereas the end-point (48) of the side mesh lattice wing members points downward in a manner substantially parallel to the centerline. The distance, “L” in FIG. 4E, between the points (47 and 48) defines the axial length of the side mesh lattice wing member. In one embodiment, the distance L for the first side mesh lattice wing member is longer than that for the second side mesh lattice wing member.

In an alternate embodiment, at least a portion of the side mesh lattice wing member (42a or 42c) is bent upward at a bending point whereas the end-point of the side mesh lattice wings points upward in a manner substantially parallel to the centerline and toward the same direction of the pushed-up wire-frame structure (not shown). In one embodiment, the side mesh lattice wing member is configured and slightly curved along the venous wall (that is, concavely upward or downward when viewed from outside) to appropriately and intimately contact or anchor at the venous wall after implantation. In one embodiment, the wire-frame structure is mounted with at least one leaflet to provide a unidirectional fluid flow as a single-leaflet or multiple-leaflet venous valve device.

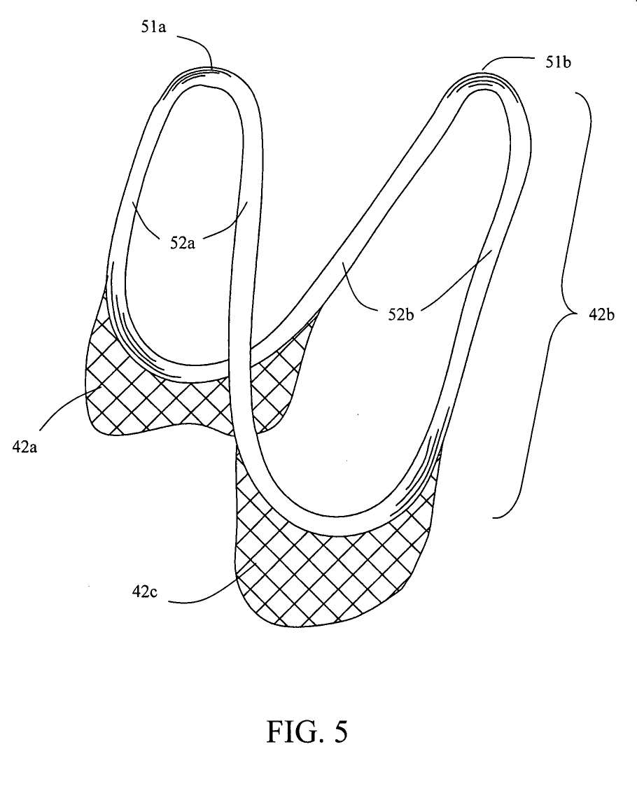

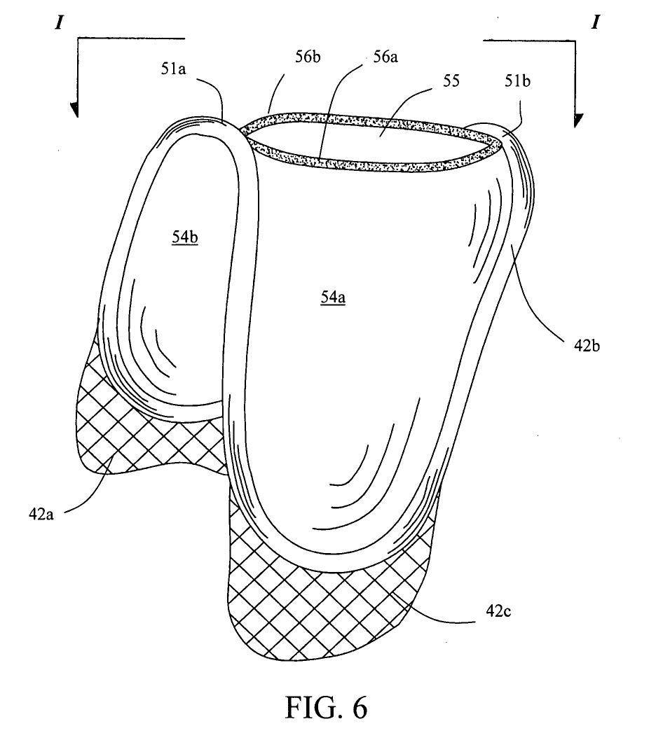

FIG. 5 shows a perspective view of the self-expandable wire-frame or venous device of the present invention, including two anchoring support members, made of the disclosed process from a one-piece sheet of biocompatible material. In one embodiment, the wire-frame is for mounting the valve leaflets along its central wire member (42b) and has two mesh-like support structures (42a and 42c) for holding (that is, anchoring) the venous valve device in place against the wall of a venous vessel. The lower portion of the central wire-frame member (42b) adjacent to the mesh-lattice support structures (42a or 42c) constitutes a base. The base and commissar points are parallel to an axial line of the vein after implantation, whereas the base is proximal to the commissar points. FIG. 6 shows a perspective view of a venous valve device with leaflets (54a and 54b) securely attached to the wire-frame. In one embodiment, the attachment suture does not interfere with leaflet movement or cause wear and abrasion of the leaflets.

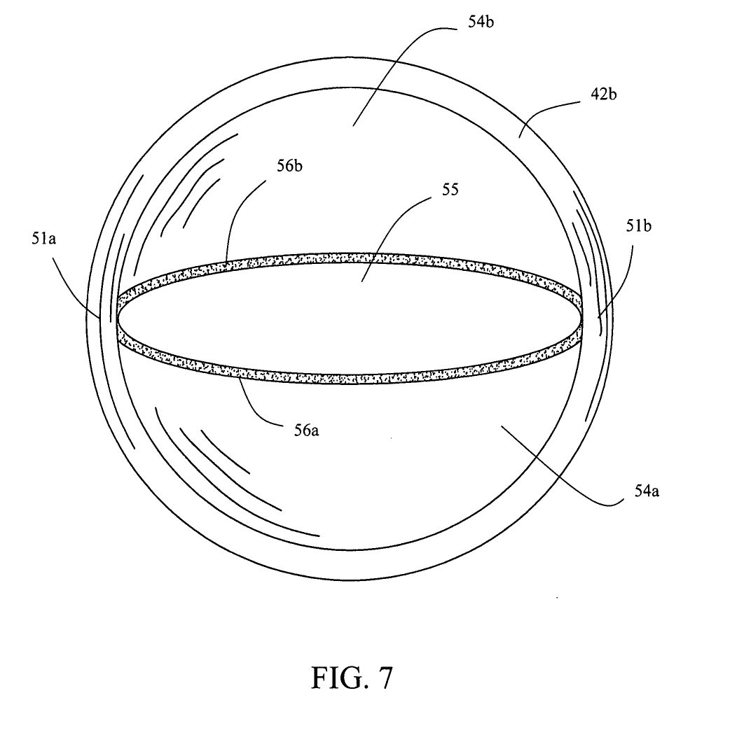

The free margin or coapting edges (56a and 56b) of the leaflets (54a and 54b, respectively) approach each other to prevent blood back-flow in a close mode. In an open mode, the opening (55) between the free edges allows blood to flow through with minimal resistance. A first end of the first free edge (56a) joins the first end of the second free edge (56b) at the first commissar (51a) whereas a second end of the first free edge (56a) joins the second end of the second free edge (56b) at the second commissar (51b) of the central wire member (42b). FIG. 7 shows a top view, section I-I of FIG. 6, of the self-expandable venous valve device, including two coaptable leaflets. The cross section of the venous valve device matches the cross section of the venous vessel after appropriately deploying the venous valve device in place.

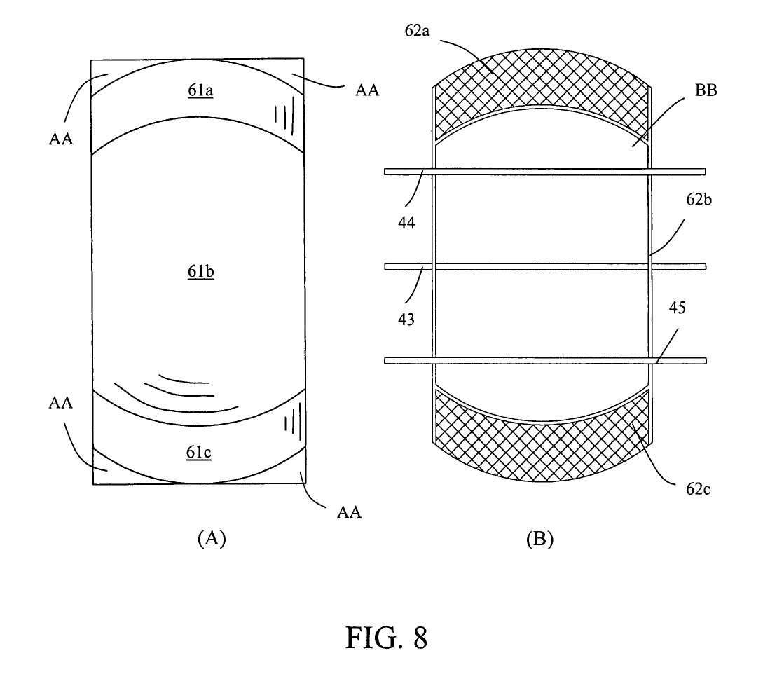

By following the similar manufacturing process as discussed above, a non-circular sheet is used in the process below. FIG. 8 shows one embodiment of forming an alternate wire-frame from a laser-cut structure in the manufacturing process of a venous device. By using a laser instrument or a micro-machine, the sheet is cut so the area “AA” is cut off and disposed of as shown in FIG. 8A. The remaining sheet contains a first wing member (61a), a second wing member (61c) and a central member (61b) that connects the first and the second wing members. The central member is a non-circular shape.

Next, by using a laser instrument or a micro-machine, the first and second wing members are cut to show mesh, mesh like, scaffold, or stent-like structure with struts and are coded ‘first wing mesh member’ (62a) and ‘second wing mesh member’ (62c) with bending flexibility and supporting/anchoring function (shown in FIG. 8B). The central portion of the central member is cut so the area “BB” is cut off and the central member shows a wire like structure that is coded ‘central wire member’ (62b).

By following the same steps as illustrated in FIG. 4, the central wire member can be shaped to show a seamless wire-frame made of the disclosed process from a one-piece sheet. The wire-frame can be used as a venous device or as a component for a venous valve device. In one embodiment, the wire-frame is for mounting two valve leaflets along its central wire member (62b) and has two support mesh-like structures (62a and 62c) for holding or anchoring the venous valve device in place against the wall of a venous vessel and prevent any blood regurgitation or leakage between the device and the wall.

Some aspects of the invention relate to an implantable venous filter device made of a sheet of biocompatible material, the device comprising: a longitudinal wire-frame structure and a base, wherein the wire-frame structure is a continuous seamless wire loop, wherein the wire-frame structure comprises a filter mechanism, the base of the wire-frame structure being radially inwardly compressible; and plural anchoring mesh (lattice) wing members spaced apart and located on the wire-frame structure (more specifically, plural anchoring mesh or lattice wing members spaced apart and connected to the base of the wire-frame structure), wherein each mesh lattice wing member and the wire-frame are integral parts from the same sheet of biocompatible material. The following patents disclose examples of such venous filter systems that can be used in manufacturing the present venous filter device: U.S. Pat. No. 5,895,398; U.S. Pat. No. 6,692,508; and U.S. Pat. No. 7,235,061.

The implantable venous valve device of the present invention is unique in that it does not attempt to mimic the form of a native vein valve, but rather, replaces the function of a native vein valve via a percutaneous deployment route. Specifically, the radial collapsing and expanding steps of the device during the delivery/deployment phase mostly involve the central wire-frame member (42b). In other words, the side mesh lattice wing members or mesh-like support structures (42a, 42c) is less radially collapsible/expandable as compared to the central wire-frame member. However, in one embodiment, the side mesh lattice wing member is somewhat more longitudinally collapsible or more flexible in the martensitic state than in the austenitic state. To facilitate passage from the delivery apparatus or sheath, the shape memory device is maintained in a collapsed configuration inside a delivery apparatus, where it is cooled by a saline solution to maintain the device below its transition temperature. The cold saline maintains the temperature-dependent device in a relatively softer condition as it is in the martensitic state within the apparatus.

In one preferred embodiment, the ratio of the wing-member axial length (“L” as shown in FIG. 4E) of all side mesh lattice wing members to the wire-frame axial length (“H” as shown in FIG. 4E) is sized between about 0.1 to 10. To maintain a patent and continuous inflow in a vein, particularly in a vena cava venous system, the ratio is sized and configured above 1.0, preferably between about 1.01 and 10.0, and most preferably between about 1.1 and 3.0. The side wing member (42a 0r 42c) has a thickness and a dimension of wing depth “D” and wing width “W”. The wing depth is defined as the length of the end mesh (49a or 49b), whereas the wing width is defined as the length between the two end meshes (49a and 49b). In another embodiment, the ratio of the wing depth to the wing width is sized between about 0.5 and about 10, preferably between about 1.0 and 10. To minimize any interference of the side wing mesh lattice member from blood flow after implantation, the ratio of the wing depth to the wing width is preferred to be between about 2.0 to 10.

Wire-Frame of Biological Origin for Venous Device

Certain biomaterial of biological origin shows shape memory properties, for example, a crosslinked chitosan scaffold or implant. U.S. patent application publication no. 2007/0014831, entire contents of which are incorporated herein by reference, discloses crosslinked collagen-containing or chitosan-containing biological devices which have shown to exhibit moisture memory and controlled, predetermined biodegradation. “Moisture memory” was herein defined a property of a device comprising a first configuration in a wet moisture state under neither external restriction nor compression, the device comprising a second configuration in a dry state under a predetermined confinement, such as compressed to be loadable in a delivery catheter, and the device reversing to the first configuration after contacting moisture when deployed from the delivery catheter in a blood vessel.

By ways of illustration, a spiral pre-product was crosslinked with a polyepoxy compound, such as ethylene glycol diglycidyl ether, or a polyepoxy compound containing at least one ether group. The device crosslinked with ethylene glycol diglycidyl ether crosslinker exhibits a first shape at a wet state, re-configurable to a second shape at a dry state, and reversible to the first shape after contacting moisture. In another embodiment, the biological material may be selected from a group consisting of collagen, gelatin, elastin, chitosan, NOCC, chitosan-alginate complex, and combinations thereof.

In one embodiment, it is contemplated that an implantable venous valve device comprises a longitudinal thread frame structure having a base (such as the one shown in FIG. 5), wherein the thread frame structure is a continuous seamless thread loop, the base of the thread frame structure being radially inwardly compressible (without re-joining), wherein the thread frame structure is optionally mounted with at least one leaflet to provide a unidirectional fluid flow; and plural anchoring mesh or lattice wing members spaced apart and connected to the base of the thread frame structure, wherein each mesh lattice wing member and the thread frame are integral parts from crosslinked chitosan-containing or collagen containing biomaterial. In one embodiment, to provide rigidity to the thread frame and the anchoring members, the biomaterial is substantially fully crosslinked, for example, a degree of crosslinking above 90%. preferably above 95%.

Venous Valve with Multiple Leaflets

Some aspects of the invention relate to a process of manufacturing the venous device comprising: (a) providing the sheet of biocompatible material; (b) cutting the sheet to form a plurality of mesh lattice wing members and a central wire member that connects the plural mesh lattice wing members on a plane, wherein the central wire member has at least two wire sections being detached from the plural mesh lattice wing member; (c) forming an individual wire-frame configuration on each of the at least two wire sections by pushing upward a central part of each wire section while holding steady the mesh lattice wing members on the plane; and (d) bending the mesh lattice wing members to become substantially parallel to a direction of the wire-frame configuration.

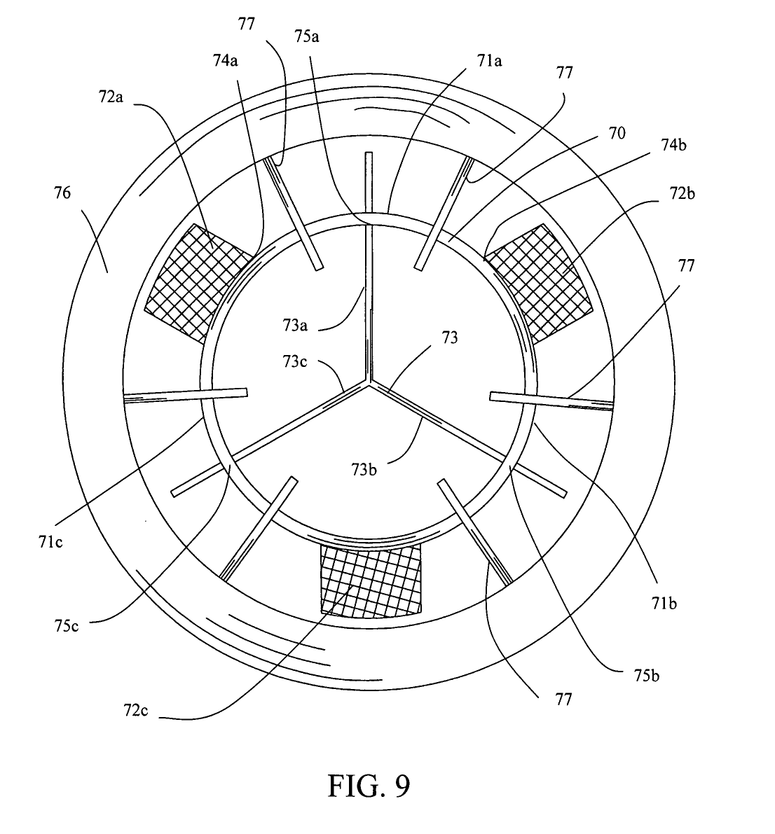

FIG. 9 shows the first step of forming a preferred wire-frame from a laser-cut structure in the manufacturing process of a venous device. Alternately, FIG. 9 shows a manufacturing process for making a three-leaflet venous valve starting from a flat sheet. The laser cut scaffold structure (70) comprises three spaced-apart supporting mesh members (72a, 72b, 72c) that are joined to one another with each of three curved connecting wires (71a, 71b, 71c). In one embodiment, the innermost surface of the scaffold structure (70) is approximately circular, when viewed from top of the structure. To bend the connecting wires to form the wire-frame shape for mounting leaflets, a set of bending tool is used. The bending tool comprises a stationary base (76) and a pushing tribar (73) that has three equally angle-spaced rigid bars (73a, 73b, 73c) at 120° apart. In one embodiment, the pushing tribar (73) has three unequally angle-spaced rigid bars (not shown).

Each of the rigid bars is placed at about the middle point (75a, 75b, 75c) of the connecting wires (71a, 71b, 71c, respectively). For example, the middle point (75a) is located at the middle point between the edge points (74a and 74b). The stationary base (76) comprises a base ring, onto which six straight rigid bars (77) at a plane are placed and pointed toward the center of the base ring. The six rigid bars (77) are spaced apart to hold each of the connecting wires steady when the pushing tribar is pushed upward. In one embodiment, any two rigid bars of the stationary base are placed onto a connecting wire with equal distance from the respective tribar.

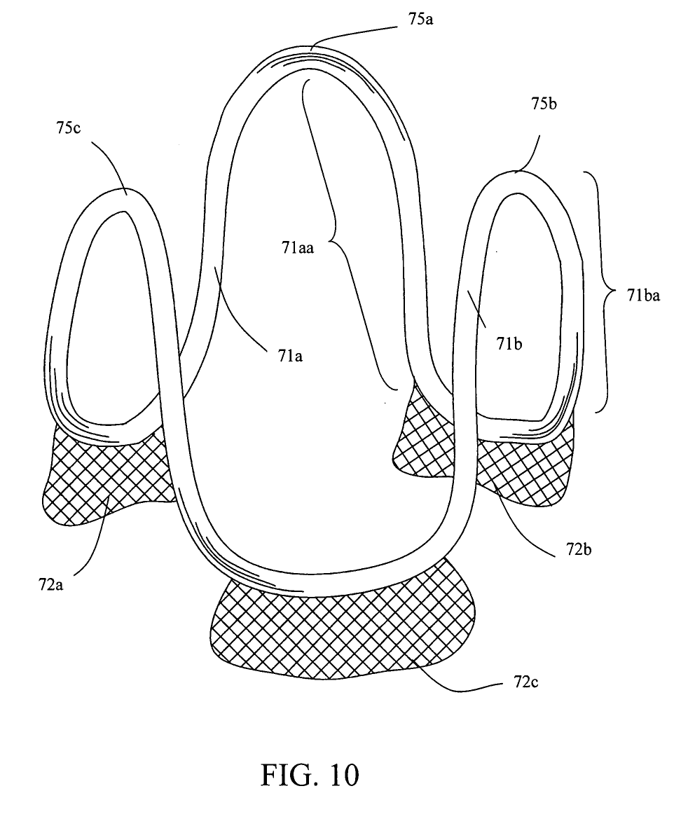

By following the similar steps as shown in FIG. 4, a wire-frame (79) with three commissars is manufactured. FIG. 10 shows a perspective view of the self-expandable venous device of the present invention, including three anchoring support members. The wire-frame (79) can be further mounted with three tissue leaflets. By ways of illustration, a first leaflet can be securely mounted from a first commissar point (75a), along with a first wire portion (71aa) of the first connecting wire (71a), a wire portion of the first mesh support member (72b) and the first wire portion (71ba) of the second connecting wire (71b), and ends at the second commissar point (75b). Each leaflet will have a free margin or coapting edge, which may be a straight line, follow a curvature, or is configured according to the disclosed equation of the present invention.

Edge Profile of the Venous Valve Leaflet

As discussed earlier, the free margin or coapting edge of the valve can be approximated with a non-contact laser or water-jet cut following equation no. 1 and/or equation no. 2. Some aspects of the invention relate to a venous valve device having a plurality of leaflets, the free margin or coapting edge of the leaflet is approximated with equation no. 1 or equation no. 2.

Some aspects of the invention relate to an implantable venous valve device made of a sheet of biocompatible material, the device comprising: a longitudinal wire-frame structure having a base, wherein the wire-frame structure is a continuous seamless wire loop (without soldering, welding, or re-joining), wherein the wire-frame structure is mounted with at least one leaflet to provide a unidirectional fluid flow; and plural anchoring mesh (lattice) wing members spaced apart and connected to the base of the wire-frame structure, wherein each mesh lattice wing member and the wire-frame are integral parts from the sheet of biocompatible material (without gluing, soldering, welding, or any re-joining operation).

Delivery of Venous Valve Device

The longitudinal wire-frame structure portion of the venous valve device is preferably adjustable between an introducing form, wherein the device is suitable for introducing into a blood vessel, and an expanded form suitable for placing the venous valve (flow stoppage element) within a blood vessel at the desired working location thereof, and most preferably has such a form as to have substantially the same length when occupying its introducing form as when occupying its expanded form. Accordingly, the device can be effectively introduced to a pre-desired location within a blood vessel, whereafter it is expandable to take up its working form. In one embodiment, the venous valve device is delivered into the leg vein at a failed vein sinus. The wire-frame structure opens up (expands radially) right below the agger in order to keep the inlet flow aspect wide open and prevent the failure of thrombosis because of narrowed inflow cross-sectional area. In one embodiment, the venous valve device of the present invention is positioned and secured in place by the anchoring function of the plural mesh latticed wing members.

The venous valve device of the present invention is sized and configured for delivery via a 4-French to 60-French (3-French equals 1 mm) sheath or delivery apparatus. The implant site and route may include any target region or area in the venous system that a percutaneous delivery device (a catheter, a wire, a trocar, a cannula, a sheath, and the like) may conveniently get access to. The current venous device, with or without valve leaflets, may be self-expanding, requiring a sheath for delivery. With a fixed annulus, the current venous valve device has sizes for 1.5-mm up to 18-mm annulus after expansion. The device can be delivered using the retrograde/transfemoral approach or transarterially. The venous device is retrievable or repositionable after the device is deployed. One optional method is to apply cold saline or Peltier effect to lower the temperature onto the Nitinol wire-frame so the wire-frame is radially collapsible for retrieval inside a retrievable sheath.

The delivery system must reliably disengage from the implanted venous device (including venous valve device, venous filter device, and the like), and be able to be removed from the vein and out of the body of the valve recipient in a straightforward and reliable manner. A percutaneous venous device delivery system for a self-expanding venous device additionally typically allows release of the self-expanding device after the self-expanding device is positioned in the desired location in its target. The following patents disclose examples of such delivery systems that can be used in delivering the instant device: U.S. Pat. No. 5,332,402; U.S. Pat. No. 5,397,351; U.S. Pat. No. 5,607,465; and U.S. Pat. No. 5,855,601.

The venous device of the present invention (without a valve leaflet as shown in FIG. 5 or with at least one valve leaflet as shown in FIG. 6) is preferably composed of a shape memory material, such as a nickel-titanium alloy commonly known as Nitinol, so that in its memorized configuration it assumes the desired expanded shape. This shape memory material characteristically exhibits rigidity in the austenitic state and more flexibility in the martensitic state. To facilitate passage from the delivery catheter or sheath, the shape memory device is maintained in a collapsed configuration inside a delivery sheath, where it is cooled by a saline solution to maintain the device below its transition temperature.

The cold saline maintains the temperature dependent device in a relatively softer condition as it is in the martensitic state within the sheath. This facilitates the exit of device from the sheath as frictional contact between the device and the inner wall of the sheath would otherwise occur if the device were maintained in a rigid, i.e. austenitic, condition. When the device is released from the sheath to the target site, it is warmed by body temperature, thereby transitioning in response to this change in temperature to an austenitic expanded condition. On being placed at its desired position within the blood system, the device, once the memory metal has achieved a particular predetermined temperature, will expand in order to assume its working form as shown in FIG. 5 or FIG. 6.

U.S. Pat. No. 7,041,128, entire contents of which are incorporated herein by reference, discloses a delivery catheter having a tubing sheath with a stopcock to control saline infusion through the catheter to maintain the venous device in the cooled martensitic collapsed configuration for delivery. The outer sheath of the delivery catheter slides with respect to the catheter shaft to expose the venous device. An optional guidewire port enables insertion of a conventional guidewire (not shown) to guide the delivery catheter intravascularly to the target site. A conventional access or introducer sheath (not shown) would be inserted through the skin and into the access vessel, and the respective delivery catheter would be inserted into the access vessel through the introducer sheath.

U.S. Pat. No. 6,807,444, commonly owned by the co-inventors of the present invention, entire contents of which are incorporated herein by reference, discloses a probe arrangement comprising two elements of different electromotive potential conductively connected at a probe junction, and passing an electrical current through the elements to reduce or raise a temperature of the probe junction in accordance with the Peltier effect. The probe arrangement is suitable for releasably connecting to the venous valve device in a delivery catheter and for reducing the junction temperature to maintain the venous device in the cooled martensitic collapsed configuration for delivery.

From the foregoing description, it should now be appreciated that a novel and unobvious venous device using decellularized pericardium mounted on a self-expandable wire-frame as a medical device has been disclosed. While the invention has been described with reference to a specific embodiment, the description is illustrative of the invention and is not to be construed as limiting the invention. Various modifications and applications may occur to those who are skilled in the art, without departing from the true spirit and scope of the invention.

What is claimed is:

1. An implantable venous device made of a sheet of biocompatible material, comprising:
a longitudinal wire-frame structure having a base, wherein the wire-frame structure is a continuous seamless wire loop, the base of the wire-frame structure being radially inwardly compressible; and
plural anchoring mesh lattice wing members spaced apart and connected to the base of the wire-frame structure, wherein each mesh lattice wing member and the wire-frame are integral parts from said sheet of biocompatible material.

2. The venous device of claim 1, wherein the wire-frame structure is mounted with at least one leaflet to provide a unidirectional fluid flow.

3. The venous device of claim 2, wherein the leaflet is made of a polymer membrane.

4. The venous device of claim 2, wherein the leaflet is a tissue leaflet.

5. The venous device of claim 2, wherein the leaflet is a decellularized tissue leaflet.

6. The venous device of claim 2, wherein the leaflet is a crosslinked tissue leaflet.

7. The venous device of claim 2, wherein the leaflet is a crosslinked tissue leaflet with a crosslinking agent of epoxy compounds.

8. The venous device of claim 2, wherein the leaflet is a crosslinked pericardium tissue leaflet.

9. The venous device of claim 2, wherein the leaflet is made of a crosslinked pericardium, wherein the pericardium is selected from a group consisting of bovine pericardium, equine pericardium, porcine pericardium, ovine pericardium, caprine pericardium, and kangaroo pericardium.

10. The venous device of claim 1, wherein the wire-frame structure comprises a filter mechanism.

11. The venous device of claim 2, wherein the leaflet is a tissue leaflet made of a process comprising steps of: providing a tissue sheet having cells and extracellular matrix; subjecting said sheet to a solution containing bile acid or bile salts that effect the solubilization of cell membranes of the cells present in said tissue sheet; removing said solubilized cell membranes by flushing the tissue sheet with filtered water or saline; and treating said tissue sheet with a crosslinking agent.

12. The venous device of claim 11, wherein the bile acid is cholic acid or deoxycholic acid.

13. The venous device of claim 11, wherein the bile salts are glycocholate or deoxycholate.

14. The venous device of claim 11, wherein the process further comprises dehydrating said decellularized tissue.

15. The venous device of claim 11, wherein the process further comprises soaking said decellularized tissue in glycerol or glycerol-alcohol mixture.

16. The venous device of claim 11, wherein the process further comprises lyophilizing said decellularized tissue.

17. The venous device of claim 1, wherein the wire-frame is made of a shape memory Nitinol alloy.

18. The venous device of claim 1, wherein said anchoring mesh lattice wing members are neither radially inwardly compressible nor outwardly expansible.

19. The venous device of claim 1, wherein a ratio of the wing-member axial length to the wire-frame axial length is between about 1.01 and 10.

20. The venous device of claim 1, wherein a process of manufacturing said device comprises: (a) providing the sheet of biocompatible material; (b) laser-cutting the sheet to form the plurality of mesh lattice wing members and a central wire member that connects the plural mesh lattice wing members on a plane, wherein the central wire member has at least two wire sections being detached from said plural mesh lattice wing member; (c) forming an individual wire-frame configuration on each of the at least two wire sections by pushing upward a central part of each wire section while holding the mesh lattice wing members on the plane; and (d) bending the mesh lattice wing members to be substantially parallel to a direction of said wire-frame configuration.
